(19) United States
(12) Patent Application Publication
(10) Pub. No.: US 2003/0083324 A1
(43) Pub. Date: May 1, 2003

(54) Photosensitizing ointment

(75) Inventor: Jorg G. Moser, Dusseldorf (DE)

Correspondence Address: CERAMOPTEC INDUSTRIES, INC., 515 Shaker Road, East Longmeadow, MA, 01028, US

(73) Assignee: CeramOptec Industries, Inc.

(21) Appl. No.: 10/282,885

(22) Filed: Oct. 29, 2002

Related U.S. Application Data
(63) Continuation-in-part of application No. 09/636,495, filed on Aug. 11, 2000.

(51) Int. Cl. A61K 31/555; A61K 31/409

(52) U.S. Cl. 514/185; 514/410

(57) ABSTRACT

A system and a method using photodynamic therapy for treatment of epithelial diseases are provided, wherein the photosensitizers used have enhanced selectivity for the affected region so that the treatment has reduced or no side effects. Selectivity is achieved by avoiding systemic application of the photosensitizer and by topically applying the photosensitizers with certain carriers. Compositions of hydrophilic medical or cosmetic carriers like ointments, creams or lotions can be used. Hydrophobic photosensitizers such as bacteriopheophorbide and its derivatives are preferred because of their ability to penetrate tissue and to distribute evenly, as well as their low threshold of phototoxicity. After the phototoxic sensitizer has been administered to the afflicted tissue, the tissue is irradiated with an appropriate radiation source, such as sunlight or a radiation source emitting a defined wavelength like a diode laser. Deeper penetration of the radiation may be achieved with longer wavelengths (700-800 nm), which are in the red part of the spectrum. The photosensitizing agent can be topically applied easily and repeatedly, and thus the system is especially useful for treating diseases like psoriasis, where frequent and repeated treatments may be necessary. A method of photodynamic therapy for epithelial diseases is also provided, which comprises the steps of: (a) applying topically a therapeutically effective amount of the photosensitizer like bacteriopheophorbide or a bacteriopheophorbide derivative to an area afflicted by an epithelial disease or an infection, and (b) exposing the treated area to radiation to photoactivate the photosensitizer to produce a cytotoxic response in the afflicted area.

### REFERENCE TO RELATED CASE

This application is a continuation in-part of co-pending U.S. patent application Ser. No. 09/636,495 filed on Aug. 11, 2000 by Jörg G. Moser, inventor, entitled “Photosensitizing Ointment”, and incorporated by reference herein.

### BACKGROUND OF THE INVENTION

1. Field of the Invention

The present invention relates to photodynamic therapy of epithelial diseases, such as tumorous skin malignancies, psoriasis, and bacterial infections in wounds. The present invention also relates to topical application of bacteriopheophorbides in the course of photodynamic therapy of epithelial diseases.

2. Information Disclosure Statement

The use of photosensitizers in treatment of hyperproliferative diseases such as tumors is well-known. The method, called photodynamic therapy (PDT), uses non-toxic, photosensitizing drugs in combination with non-hazardous light irradiation to destroy the malignant tissue or cells.

When cells containing photosensitizers are exposed to radiation of the respective absorbed wavelength that activates the photosensitizers, cytotoxicity is induced by intracellular formation of singlet oxygen, a short-lived, highly reactive state of the oxygen molecule. The photosensitizers used in PDT need to have characteristics of high affinity and selectivity for the malignant tissue as well as a high quantum yield for singlet oxygen production. Porphyrins have a high quantum yield to form an excited triplet state. The difference between the energies of the triplet state and their singlet ground state makes porphyrins good energy donors to transfer their energy to the ground state of oxygen to form the highly reactive singlet oxygen.

The wavelengths that are ideally used in PDT lie in the “photodynamic window” (650-850 nm) where biological chromophores in humans normally do not absorb. The minimum wavelength of 650 nm is given because of the absorption properties of biological chromophores, e.g. heme in hemoglobin, and light scatterers like melanin. The maximum wavelength is due to the energy transfer process from the sensitizer triplet to triplet oxygen to yield the reactive singlet oxygen. Sensitizers absorbing at higher wavelengths show significantly reduced yields of singlet oxygen. Since longer wavelengths penetrate deeper into the tissue, it is desirable that the photosensitizers are activated by the longer wavelengths within the “photodynamic window” for the removal of a whole tumor.

Moreover, a photosensitizer should be characterized by a low “threshold dose,” which is the minimum energy dose at which a photosensitizer becomes phototoxic. One of the phototoxic effects is a low power “overshot,” which is defined as the non-desirable activation of cell proliferation at low power densities.

The use of PDT has been described for therapies of inner organ tumors as well as skin malignancies. Epithelial diseases (epidermal and mucosal diseases) are a major health problem. Nearly everybody suffers from epithelial diseases several times during his or her life. Examples of hyperproliferative epithelial diseases not only include cutaneous tumors (basal cell carcinoma, squamous cell carcinoma, melanoma), but also include psoriasis, virus-caused diseases (warts, herpes simplex, condylomata acuminata), premalignant and malignant diseases of the female genital tract (cervix, vagina, vulva), and premalignant and malignant diseases of mucosal tissues (oral, bladder, rectal).

The most common skin disease is psoriasis. The causes of psoriasis probably lie in genetic factors which cannot be cured by the medical tools available today. Therefore, the therapy is reduced to control the symptoms. The treatment often has to be repeated during the lifetime of a patient. A variety of therapies currently used to treat psoriasis include dialysis, chemotherapy (topical and systemic), and PDT (topical and systemic). Topical chemotherapy is probably the most widely used, employing agents such as retinoids, anthralin, corticosteroids, and antimetabolites. At present, the most severe cases of psoriasis are treated with systemic phototherapy, e.g. the use of oral methoxypsoralen and long-wave ultraviolet light (PUVA). Clinically, PUVA has remained relatively effective for the majority of patients, and short-term side effects, such as widespread severe erythema, have been tolerable for the severely afflicted patients. The therapeutic mechanism of PUVA is proposed to be based on the binding of psoralens to the DNA of the afflicted cells. The binding leads to inhibition of DNA synthesis and consequent blocking of cell divisions. While the inhibition of DNA synthesis may be the desirable outcome of psoriasis therapy, there are concerns that the direct changes in the DNA structure and function by PUVA may have potential carcinogenic and mutagenic effects. Thus, while methods employing PUVA have shown some promises in the treatment of epithelial diseases so far, it is desirable to develop new therapeutic strategies that are equal or even more effective without undesirable side effects, such as erythema over unafflicted areas of a patient, and the potential carcinogenic effect of the treatments.

The treatment of psoriasis employing photosensitizers like hematoporphyrin derivatives (HPD) that generate singlet oxygen for the destruction of the malignant cells has been described. Also, the therapeutic potential of HPD for tumors was demonstrated, and several clinical trials using HPD photoirradiation therapy have been reported in patients with cutaneous or subcutaneous malignant tumors. However, the absorption maximum of HPD lies at wavelengths where biological chromophores can absorb, and therefore the penetration depth of the irradiation is not sufficient to activate the phototoxic dye for a complete removal of the malignant tissue. Moreover, due to the high systemic doses necessary to achieve therapeutic levels of the photosensitizer at the tumor sites, high concentrations are detected in other, non-malignant organs. However, Bacteriopheophorbide and its derivatives, e.g. 13-OH-bacteriopheophorbide, are dyes that meet the requirements for PDT much better than the dyes mentioned above. The maximum optical absorption of these compounds is located well into the longer wavelength part of the “photodynamic window” so that the irradiation used penetrates deep enough into the tissue for the removal of the whole tumor. The high phototoxicity of the bacteriopheophorbides makes the therapy more efficient and thereby makes dosage reduction of the applied sensitizer possible. A reduced dosage of sensitizers reduces side effects of the therapy.

Although bacteriopheophorbide and its derivatives are more efficient, they still have significant disadvantages when they are used systemically. One reason is that the patient's entire skin is photosensitized. The advantage of PDT, compared with the commonly used chemotherapy or radiotherapy, is the selectivity for the treatment site through administration of radiation only to a limited space, and this advantage cannot be easily achieved at the skin. Sun light reaching a patient's skin is sufficient to activate the phototoxicity of the sensitizer at least in the outer layers of the skin, which causes widespread severe erythema. Therefore, the whole-body photosensitivity after systemic injection requires the patient to avoid direct sunlight or prolonged contact with bright artificial light for several weeks. Since many epithelial diseases affect only small and superficial areas, it is unreasonable and inconvenient to treat such patients with a systemic medication and expose them to these side effects. One solution to this problem would be to develop a composition with a photoactive drug that is effective when applied topically. Topically applied drugs provide an ideal method of localizing the effects of the drug, since they need to be applied only to the afflicted tissue. However, many systemically active drugs are ineffective in topical formulations. It is especially hard for them to penetrate through the epidermis which is designed to protect the organism from foreign substances. The barrier function of the skin is achieved by its special cell types and assemblies. Keratinocyte is a type of cell that constitutes the epidermis, and corneocytes are linked by and embedded in lipid layers in the uppermost layer (stratum corneum) of the skin. Due to this structure, only hydrophobic substances, like bacteriopheophorbides, can penetrate effectively through the epidermis. This penetration is a prerequisite for the action of topically administered therapeutic agents.

The frequent occurrence of epithelial diseases makes an effective treatment without serious side effects necessary. Although hydrophobic substances such as bacteriopheophorbides do not combine well with hydrophilic substances, the present invention demonstrates the unexpected finding that a combination of hydrophobic photosensitizers and hydrophilic topical carriers can prove effective and thus can address the need for an effective topical PDT application to reduce the side-effects described above.

### BRIEF SUMMARY AND OBJECTS OF THE INVENTION

It is an object of the present invention to provide an effective therapeutic photodynamic method for the treatment of epithelial diseases, such as skin tumors, psoriasis, and infections of wounds.

It is another object of the present invention to provide a topical composition for the treatment of epithelial diseases, and at the meantime to avoid the serious side effects associating with the systemic administration of photosensitizers.

It is yet another object of the present invention to provide an improved photodynamic therapeutic method for the treatment of epithelial diseases by using improved phototherapeutic agents like bacteriopheophorbide and its derivatives.

Briefly stated, the present invention provides a system and a method using photodynamic therapy for the treatment of epithelial diseases, wherein the photosensitizers used have enhanced selectivity for the affected region so that the treatment has less or no side effects. The selectivity is achieved by avoiding the systemic application of the photosensitizer as well as by using topical application of the photosensitizers with certain carriers. Compositions of hydrophilic medical or cosmetic carriers like ointments, creams or lotions can be used as a carrier. Hydrophobic photosensitizers such as bacteriopheophorbide and its derivatives are preferred photosensitizers because of their abilities to penetrate the tissue and to distribute evenly, as well as their low threshold of phototoxicity. After the phototoxic sensitizer has been administered to the afflicted tissue, the tissue is irradiated with an appropriate radiation source, which can be sunlight or a radiation source emitting a defined wavelength like a diode laser. A deeper penetration of the radiation may be achieved by using longer wavelengths (700-800 nm), which are in the red part of the spectrum. The present invention provides a system that the photosensitizing agent can be topically applied easily and repeatedly, and thus especially useful for the therapy of a disease like psoriasis, where frequent and repeated treatments may be necessary. The present invention also provide a method of photodynamic therapy for epithelial diseases, which comprises the steps of: (a) applying topically a therapeutically effective amount of the photosensitizer like bacteriopheophorbide or a bacteriopheophorbide derivative at the treating area, which is afflicted by a epithelial disease or an infection, and (b) exposing the treated area of skin to radiation so that the radiation photoactivates the photosensitizer to produce a cytotoxic response in the afflicted area.

### DETAILED DESCRIPTION OF PREFERRED EMBODIMENTS

For topical administration, a photosensitizer has to have such properties so that it can penetrate through the epidermis into the skin and distribute evenly within the diseased tissue. Alternatively, certain additives have to fulfill these functions while supporting the therapeutic action of the photosensitizers. The present invention is applicable to the treatment of epithelial diseases, such as hyperproliferative epithelial diseases, e.g. melanoma, psoriasis, and infections of wounds in animals (such as mammals, and particularly in humans). The present invention meets the special requirements for treating epithelial diseases, wherein the property of the carrier used, the skin penetration depth of the drug, and the distribution of the drug are critical when the drug is topically administered. Despite the fact that hydrophilic and hydrophobic substances do not combine well, it was found, surprisingly, that a composition consisting of a hydrophobic photosensitizer and hydrophilic carrier would make an effective topical composition.

The more hydrophilic the carrier is, the more efficient the penetration of the drug through the epidermis is. The use of hydrophilic carriers enhances the efficiency of penetration of these drugs through the epidermis without the need for additional penetrants that could irritate the skin. There are several reasons for this unanticipated result.

The unique structure of the mammalian epidermis, which is designed to protect the organism from substances of the environment, makes penetration into the skin impossible for many drugs. The barrier function is achieved by corneocytes linked to and embedded in lipid layers. Such barrier can be penetrated only by hydrophobic substances, such as bacteriopheophorbides. Since bacteriopheophorbide and its derivatives meet the special requirements for penetration through the epidermis, it is especially well suited for topical administration.

Because a hydrophilic carrier cannot penetrate the epidermis, a hydrophobic drug used in conjunction with such a carrier enters the epidermis at a higher effective concentration. The photosensitizer is more efficiently administered through the skin to a desired treatment site because no other compounds enter through the skin's barrier layer. Efficiency is also enhanced because the hydrophobic photosensitizer tends to be repelled by a hydrophilic carrier. In this way the hydrophilic carrier enhances penetration because it, in essence, helps to force the hydrophobic photosensitizer through the skin barrier. This result is surprising due to the conflict in the characteristics of hydrophobic photosensitizers and hydrophilic carriers. Although hydrophilic topical compositions, because they do not tend to penetrate the skin, are often used as lubricants or protectants, it has been found that hydrophilic carriers are useful in a skin penetration composition such as the present invention because they enhance the chemical driving force for the hydrophobic photosensitizers to penetrate the skin.

Further benefits of the present invention arise from the fact that hydrophilic carriers are often less irritating and can be easily removed from the skin after the hydrophobic photosensitizers such as bacteriopheophorbide or derivatives are delivered.

The penetration can be ameliorated by the use of penetration enhancers, which are frequently used in cosmetics and medicine. However, since such enhancers often cause irritation of the skin, e.g. by DMSO, their exclusion is preferred. Bacteriopheophorbides and its derivatives penetrate readily through the epidermis and distribute well within the skin without penetration enhancers. Therefore, they are especially preferred as therapeutic active photosensitizers in topical administrations. Moreover, Bacteriopheophorbides and their derivatives show lower threshold of phototoxicity compared to other photosensitizers. This is important for the topical application of the drug, since low concentrations are sufficient for the therapeutic action and removal of the abnormal tissues, e.g. the whole tumor. The phototoxicity can be used to destroy cancer cells while leaving healthy cells undamaged if the application of the photosensitizer and its radiation activation is strictly localized to the malignant tissue. Moreover, healthy cells have the capacity to regenerate when they are only slightly damaged by the photodynamic therapy.

The present invention is directed to treatment of epithelial diseases, such as infection of wounds, hyperproliferative diseases like tumors and psoriasis. Since these diseases are very common, there is a clear need for an efficient therapy. The use of PDT has been shown to be efficient in the treatment of tumor as well as psoriasis. However, the systemic administration of the photosensitizer used so far has the disadvantage of photosensitizing the patient's whole skin, so that the patient has to protect himself from light for several weeks to avoid the severe side effects of this whole-body photosensitation. The present invention provides a system using topical application instead of the systemic administration of the photosensitiser, so that the phototoxic drug is confined to the afflicted tissue.

The epithelial diseases, as used herein, mean conditions of the skin that are characterized by epidermal cell proliferation, incomplete cell differentiation, or other premalignant lesions. The topical compositions of the present invention may be used to treat hyperproliferative epithelial disease, including cutaneous malignancies which occur primarily to the skin (e.g. squamous cell carcinoma, basal cell carcinoma, melanoma), metastatic, non-nodal lesions of internal malignancies present on the skin, psoriasis, viral diseases such as herpes simplex and warts, as well as bacterial infections. The success of a therapy also depends on a drug's ability to penetrate through the epidermis and distribute evenly in deeper skin layers. When topically administered, bacteriopheophorbide and its derivatives in a suitable carrier meet these requirements very well. Fischer E. in The Chlorophylls, Scheer H. ed., pp 161-162, CRC Press, Boca Raton (1991) demonstrates that bacteriopheophorbide and its derivatives, such as 13-OH-bacteriopheophorbide or metallo derivatives, are products from bacteriochlorophyll and are obtained by acid hydrolysis in acetone-sulfuric acid of bacteriochlorophyll. The optical absorption maximum of these compounds in organic solvents as well as in physiological media containing protein, e.g. cell culture media, is 762 nm, which is well located in the longer wavelength part of the “photodynamic window”. Since these longer wavelengths penetrate deeper into the tissue, the removal of the whole malignant tissue is possible. The molar absorption coefficient of these compounds is 70,000, and thereby these compounds are more efficient than others used for PDT. Moser J. G. et al. Bacteriopheophorbide esters: Sensitizers without “threshold dose?” SPIE Biomed. Optics 2078, 193-204 (1994), Moser J. G. et al. Significance of “threshold dose” for photodynamic therapy of melanotic and amelanotic tumors, SPIE Biomed. Optics 2371: 178-186 (1995), and Moser J. G. et al. Subcellular storage compartments of bacteriopheophorbide sensitizers, SPIE Biomed. Optics 2978: 532-538 (1994) demonstrate the photodynamic activity of the bacteriopheophorbides in cultured cancer cells (OAT 75, A 375 cells). Neither 13-OH-bacteriopheophorbide nor metallo derivatives have been applied to humans. However, they have been tested for their tumor selectivity and therapeutic effects in animals. The compounds are preferably efficient due to their low “threshold dose”, low overshot, and high photodynamic activity (photodynamic constant LD 90=0.5−1.0 J/cm²).

A major component of compositions for topical application is the carrier. The term “carrier” as used herein refers to carrier materials suitable for topical applications of drugs, including such materials known in the cosmetic and medical fields. Suitable carriers can be, for example, ointments, creams, or lotions. Oil-in-water emulsions, such as cold cream bases, can also be used. The topical carriers described herein also include various agents and ingredients commonly employed in dermatological and cosmetic ointments and lotions. Due to a better separation from the carrier cream, the more hydrophilic the carrier is, the more efficient the drug's penetration into the skin.

The phototoxic compounds can be mixed with the carrier in a solubilized form. The bacteriopheophorbies are poorly soluble in water, but well soluble in DMSO, methanol, acetone, and 2-methoxy ethanol. 2-methoxy ethanol shows no toxicity to human skin, especially when it is diluted with cream to <20% (w/w).

It is also preferred that a topical formulation includes a skin penetration agent. One of the commonly used skin penetration agents is DMSO, which is also a solvent for bacteriopheophorbides. Occlusion can also enhance the therapeutic effects of phototoxic dye in topical application. After the drug with the carrier is applied to the afflicted skin, a barrier is placed over the area, which prevents random passage of the topical formulation and enhances the drug's absorption into the skin.

The appropriate dosage of a phototoxic dye depends upon various factors, such as the nature of the disease, the stage of the disease, and the condition of the skin. In topical applications, the ultimate dosage delivered to the afflicted tissue depends upon factors such as concentration of the phototoxic bacteriopheophorbide in the topical carrier, the amount of the topical composition which is applied to the afflicted tissue, the number of times it is applied, and the condition of the skin. In general, a concentration of 400 μM of bacteriopheophorbide in a topical composition is suitable to obtain sufficient amounts of the dye in the skin. Generally, a period of time is allowed to elapse after administration of the phototoxic dye and before exposing the afflicted tissue to radiation. The length of time necessary varies depending upon the nature of the disease, the mode of the application, and other factors. In general, a period up to about 24 hours is appropriate. This should allow sufficient time for the dye to penetrate the skin and localize in the cells of the afflicted tissue. While it may be necessary to apply the topical compositions of the present invention only once prior to radiation, it may also be necessary to repeat the application several times prior to exposure in order to obtain sufficient quantities of phototoxic dye in the afflicted tissue. To obtain complete eradication or clearing of a particular hyperproliferative epithelial disease, it may also be necessary to repeat the entire regimen of topical or interdermal applications followed by radiation. When the diseases are chronic and treatments only relieves symptoms, e.g., psoriasis, continued maintenance therapy may be required. Because the present invention has less side effects and less cumbersome, it has the advantages over the prior arts in treating such diseases.

The irradiation is performed after infiltration of the phototoxic dye into the afflicted region of the skin. Non-damaging forms of radiation in the red region of the visible spectrum are sufficient to activate the phototoxic action of the bacteriopheophorbides. The radiation source can be sunlight or a bright lamp. For the deeper penetration into the tissue, a diode laser (762 nm emission) or a lamp equipped with a red light filter (exclusion limit<762 nm) is more specific and suitable. The irradiation should not surpass 4 mW at the depth of the tissue desired for the therapeutic action. It is known how to calculate the penetration depth using light absorption wavelengths of skin.

The present invention is further illustrated by the following examples, but is not limited thereby.

### EXAMPLE 1

Preparation of an Ointment as Carrier with the Phototoxic Bacteriopheophorbide.

Basic ointment DAC is a medical carrier for hydrophobic as well as hydrophilic drugs, and allows an effective transfer of the hydrophobic bateriopheophorbide into the skin through the hydrophobic lipid layers of the epidermis. The following composition has a final concentration of 400 μM bacteriopheophorbide, which can be varied according to the requirements of the application.

This composition should be available by prescription under the name “Bacphein-400 ointment” in future.

Basic ointment DAC . . . 80.0 G

2 mM solution of bacteriopheophorbide in 2-methoxy ethanol . . . 20.0 G

sum . . . 100.0 G

### EXAMPLE 2

Preservation of the Phototoxic Dye from Oxidation.

The ointment as formulated in example 1 is protected from oxidation by filling the ointment into a tube wrapped by a tight closure. Under these conditions, oxidation can be prevented for at least three months.

### EXAMPLE 3

Application of the Composition to the Skin and Infiltration of the Phototoxic Agent into the Skin at Certain Time Period that Generally Required between Application and Irradiation.

The infiltration of the dye can be observed by the fluorescence of the skin. For this purpose, the ointment is applied in ˜1 mm thickness to the skin and removed after a time span of 2, 4, 6, 12 or 24 hours. After application, the skin is cut by a microtome transversely, and is observed under a microscope with excitation of the fluorescence of the dye using radiation with 530 nm wavelength. The diffusion depth was found to be deep enough to cover all cancerous tissue in primary melanoma and other skin diseases like psoriasis.

Having described preferred embodiments of the invention it is to be understood that the invention is not limited to the precise embodiments, and that various changes and modifications may be effected therein by skilled in the art without departing from the scope or spirit of the invention as defined in the appended claims.

What is claimed is:

1. A composition for topical application in the photodynamic therapy of an epithelial disease comprising:
a hydrophilic topical carrier;
a therapeutically effective amount of a hydrophobic photosensitizer.

2. A composition according to claim 1, wherein said topical carrier is selected from a group: a cream, an ointment, a gel, and a lotion.

3. The composition according to claim 1, wherein said photosensitizer has an absorption maximum above 700 nm.

4. A composition according to claim 1, wherein said photosensitizer is bacteriopheophorbide.

5. A composition according to claim 1, wherein said photosensitizer is a bacteriopheophorbide derivative, preferably one having enhanced hydrophobicity.

6. A method of photochemotherapy for hyperproliferative epithelial diseases comprising the steps of:
(a) applying topically a composition according to claim 1 to an area of tissue afflicted by a proliferative epithelial disease;
(b) waiting for a time period that is necessary for said composition to infiltrate into said tissue; and
(c) exposing said tissue to radiation to photoactivate said photosensitizer to produce a cytotoxic response in said tissue.

7. A method of photochemotherapy for hyperproliferative epithelial diseases according to claim 6, further comprising before step c.) the step of:
(d) removing remnants of said composition from said skin surface by wiping, washing, and/or utilizing suitable solvents

8. A method of photochemotherapy for hyperproliferative epithelial diseases according to claim 6, further comprising the step of:
(d.) applying, where necessary, a protective layer or bandage to the sensitized skin surface for a time interval corresponding to a clearance time of said photosensitizer, so as to enable the patient to be exposed to daylight or even sunlight.

9. The method according to claim 6, wherein said hyperproliferative epithelial disease, that is being treated, is tumorous skin malignancies, melanoma and Kaposi sarcoma, psoriasis, wounds infected by gram positive bacteria, or wounds infected by gram negative bacteria.
